(19) United States
(12) Patent Application Publication
(10) Pub. No.: US 2014/0010896 A1
(43) Pub. Date: Jan. 9, 2014

(54) Composition for removing a tick and providing relief

(71) Applicant: Cynthia M. Chapman, Lone Grove, OK (US)

(72) Inventor: Cynthia M. Chapman, Lone Grove, OK (US)

(21) Appl. No.: 13/937,278

(22) Filed: Jul. 9, 2013

Related U.S. Application Data
(60) Provisional application No. 61/669,221, filed on Jul. 9, 2012.

(51) Int. Cl. A61K 36/185; A61K 33/06

(52) U.S. Cl. CPC A61K 36/185; A61K 33/06
USPC 424/698; 424/725

(57) ABSTRACT

In an embodiment of the present invention, a composition for removing a tick and providing relief therefrom comprises a solvent, a carrier fluid suitable for increasing the rate of seepage of the solvent into the host, a solubilizer suitable for increasing the solubility of the composition in water, and a solution that provides soothing and aids in healing the host.

### CROSS-REFERENCE TO RELATED APPLICATIONS

This application claims the benefit of U.S. Provisional Application Ser. No. 61/669,221, filed on Jul. 9, 2012, and is incorporated herein by reference.

### BACKGROUND

1. Field of the Invention

The present invention relates to methods and compositions for the removal of a tick. More particularly, the present invention relates to a composition that effectively aids in the proper removal of a tick while at the same time providing soothing effects and accelerating the healing process for the host.

2. The Relevant Technology

Various methods exist for the removal of a tick from a host. For many years, it was a common belief that using tweezers, petroleum jelly, hot objects, or acetone was the best way to remove a tick from the host. However, these methods had a number of drawbacks, including the fact that the tick would either regurgitate what it had eaten, or its head and cement would often remain lodged in the host—which could create infections and even lead to the transmission of tick-borne diseases. Another method is to use a solution or composition tick release aid as described in U.S. Pat. No. 5,317,041. While this method promotes proper tick removal by removing the entire tick and cement—lowering the risk of disease transfer—it fails to address a major problem: even if the tick is successfully removed, the host may still suffer from considerable irritation at the entry site and the wound may still become infected. The present invention seeks to solve these problems.

### SUMMARY OF EXAMPLE EMBODIMENTS

In an embodiment of the present invention, a composition for removing a tick and providing relief therefrom comprises a solvent, a carrier fluid suitable for increasing the rate of seepage of the solvent into the host, a solubilizer suitable for increasing the solubility of the composition in water, and a solution that provides soothing and aids in healing the host.

In one embodiment of the present invention, the solvent is comprised of limonene or d-limonene, which is most commonly found in citrus peel oil. The solution for soothing and healing being comprised of the extract of the Hamamelis virginiana (Witch-hazel shrub).

In another embodiment of the present invention, the solvent is comprised of acetone. In yet another embodiment, the solvent is comprised of ethyl acetate.

In another embodiment, the solution for soothing and healing is comprised of a class of chemicals known as the alums (AB(SO₄)₂.12H₂O).

In yet another embodiment, the composition further comprises an aromatic and antiseptic means. In one embodiment, the aromatic and antiseptic means comprises Citrus sinensis (aka Blood Orange Essential Oil).

### DETAILED DESCRIPTION OF EXAMPLE EMBODIMENTS

The following descriptions depict only example embodiments of the invention and are not to be considered limiting of its scope.

In general, an embodiment of the proposed invention relates to a composition for the proper removal of the entire tick and its accompanying cement and providing relief therefrom. In particular, an embodiment of the proposed invention comprises a solvent, a carrier fluid suitable for increasing the rate of seepage of the solvent into the host, a solubilizer suitable for increasing the solubility of the composition in water, and a solution that provides soothing and aids in healing the host.

In one embodiment of the present invention, the composition comprises about twenty-five volume percent (25%) to eighty-five volume percent (85%) solvent. The solvent being further comprised of citrus peel oil having a content of about eighty-five volume percent (85%) of limonene. While the example given contemplates using the oil of a citrus peel to obtain the limonene, it will be appreciated by those with skill in the art that an actual citrus peel is not required to be used and that any commercially available concentrate of limonene, d-limonene, or its equivalent is contemplated herein (for example, the line of FloraSolv™ products by FloraChem).

In another embodiment, the solvent comprises acetone. In yet another embodiment, the solvent comprises ethyl acetate.

In one embodiment, the composition comprises about fifteen volume percent (15%) to seventy-five volume percent (75%) of a carrier fluid. The carrier fluid being comprised of lower alcohols such as methyl alcohol, ethyl alcohol, or isopropyl alcohol, with a grade of about seventy-six percent (76%) and higher.

The composition further comprising about ten volume percent (10%) of an oil solubilizer. The oil solubilizer being comprised of Polysorbate 80 (polyoxyethylene (20) sorbitan monooleate). It will be appreciated by those with skill in the art that while Polysorbate 80 is referenced, it is in no way limiting of scope and that any commercially available oil solubilizer would suffice.

The composition further comprising about 0.44 to 2.21 volume percent of a soothing and healing solution. The soothing and healing solution being comprised of the extract of the Hamamelis virginiana (Witch-hazel shrub). Commercially available Witch Hazel solutions being comprised of about 86 volume percent of astringent. Astringents are used in the preferred embodiment because they cause mild coagulation of skin proteins, which dries, hardens, and protects the skin.

In another embodiment, the solution for soothing and healing is comprised of Alum (hydrated potassium aluminum sulfate; KAl(SO₄)₂.12H₂O) or from its class of chemicals known as the alums (AB(SO₄)₂.12H₂O). Alum is an astringent with acidic properties, meaning that it may also assist with the dissolving of the tick cement. Therefore, the volume percent of solvent used in conjunction with Alum may be lowered so as to not create irritation at the wound site.

It will be appreciated that while Witch Hazel and Alum are used in the above embodiments, any solution or substance with similar qualities may be used. Astringents are readily available in many forms, all of which are contemplated herein.

In yet another embodiment of the present invention, the composition is further comprised of an aromatic and antiseptic means. In one embodiment, the aromatic and antiseptic means comprises about 0.5 volume percent (about one and one-half tablespoons per gallon) of Citrus sinensis (a.k.a. Blood Orange Essential Oil). Blood Orange Essential Oil is considered to have anti-depressant, antiseptic, and aphrodisiac properties, with the scent being uplifting and stimulating.

In another embodiment of the present invention, the composition is diluted to about one-to-one using water so as to reduce the risk of irritation to sensitive skin, such as that of animal hosts.

What is claimed is:

1. A composition for removing a tick and providing relief therefrom, the composition comprising:
a solvent;
a carrier fluid suitable for increasing the rate of seepage;
a solubilizer suitable for increasing the solubility of the composition in water; and
a solution that provides soothing and aids in healing.

2. The composition of claim 1, wherein the solvent is comprised of citrus peel oil having a content of about 85 volume percent of limonene.

3. The composition of claim 1, wherein the solvent is comprised of acetone.

4. The composition of claim 1, wherein the solvent is comprised of ethyl acetate.

5. The composition of claim 1, wherein the carrier fluid is comprised of lower alcohols.

6. The lower alcohols of claim 5 comprising methyl alcohol, ethyl alcohol, or isopropyl alcohol.

7. The composition of claim 1, wherein the solubilizer comprises Polysorbate 80 (polyoxyethylene (20) sorbitan monooleate).

8. The composition of claim 1, wherein the solution that provides soothing and aids in healing comprises the extract of Hamamelis virginiana (Witch-hazel shrub).

9. The composition of claim 1, wherein the solution that provides soothing and aids in healing comprises Alum (hydrated potassium aluminum sulfate).

10. A composition for removing a tick and providing relief therefrom, the composition comprising:
a solvent;
a carrier fluid suitable for increasing the rate of seepage;
a solubilizer suitable for increasing the solubility of the composition in water;
a solution that provides soothing and aids in healing; and
an aromatic and antiseptic means.

11. The composition of claim 10, wherein the solvent is comprised of citrus peel oil having a content of about 85 volume percent of limonene.

12. The composition of claim 10, wherein the solvent is comprised of acetone.

13. The composition of claim 10, wherein the solvent is comprised of ethyl acetate.

14. The composition of claim 10, wherein the carrier fluid is comprised of lower alcohols.

15. The lower alcohols of claim 14 comprising methyl alcohol, ethyl alcohol, or isopropyl alcohol.

16. The composition of claim 10, wherein the solubilizer comprises Polysorbate 80 (polyoxyethylene (20) sorbitan monooleate).

17. The composition of claim 10, wherein the solution that provides soothing and aids in healing comprises the extract of Hamamelis virginiana (Witch-hazel shrub).

18. The composition of claim 10, wherein the solution that provides soothing and aids in healing comprises Alum (hydrated potassium aluminum sulfate).

19. The aromatic and antiseptic means of claim 10 comprising Citrus sinensis (Blood Orange Essential Oil).

20. A composition for removing a tick and providing relief therefrom, the composition comprising:
at least 25 volume percent of citrus peel oil;
at least 15 volume percent of a lower alcohol as a carrier fluid;
at least 10 volume percent of Polysorbate 80 (polyoxyethylene (20) sorbitan monooleate); and;
at least 0.44 volume percent of Hamamelis virginiana.
